SEQUENCE LISTING (13 sequences)

SEQ ID NO: 1 - RNA, 131 nt, Artificial Sequence
  Synthetic Construct
aacaauagaugacuuacaacuaaucggaaggugcagagacucgacgggagcuacccuaacgucaagacgaggguaaagagagaguccaauucucaaagccaauaggcaguagcgaaagcugcaagagaaug

SEQ ID NO: 2 - RNA, 116 nt, Artificial Sequence
  Synthetic Construct
aaauaauugagccuuaaagaagaaauucuuuaaguggaugcucucaaacucagggaaaccuaaaucuaguuauagacaaggcaauccugagccaagccgaaguaguaauuaguaag

SEQ ID NO: 3 - RNA, 52 nt, Artificial Sequence
  Synthetic Construct
aaaauccguugaccuuaaacggucguguggguucaagucccuccacccccac

SEQ ID NO: 4 - RNA, 16 nt, Artificial Sequence
  Synthetic Construct
gagacgcuacggacuu

SEQ ID NO: 5 - RNA, 33 nt, Artificial Sequence
  Synthetic Construct
gggagacccucgaccgucgauuguccacugguc

SEQ ID NO: 6 - RNA, 35 nt, Artificial Sequence
  Synthetic Construct
accaguggacaaucgacggauaacagcauaucuag

SEQ ID NO: 7 - DNA, 27 bp, Artificial Sequence
  Synthetic Construct
acgagtcctggactgaaacggacttgt

SEQ ID NO: 8 - RNA, 9 nt, Artificial Sequence
  Synthetic Construct
gccaccaug

SEQ ID NO: 9 - RNA, 54 nt, Artificial Sequence
  Synthetic Construct
gagggcagaggaagucuucuaacaugcggugacguggaggagaaucccggcccu

SEQ ID NO: 10 - RNA, 57 nt, Artificial Sequence
  Synthetic Construct
gcuacuaacuucagccugcugaagcaggcuggagacguggaggagaacccuggaccu

SEQ ID NO: 11 - protein, 223 aa, Artificial Sequence
  Synthetic Construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 12 - protein, 31 aa, Artificial Sequence
  Synthetic Construct
DAMKRGLCCVLLLCGAVFVSPSQEIHARFRR

SEQ ID NO: 13 - protein, 17 aa, Homo sapiens
DWTWILFLVAAATRVHS

(19) United States
(12) Patent Application Publication
(10) Pub. No.: US 2025/0051816 A1
(43) Pub. Date: Feb. 13, 2025

(54) SINGLE-POT METHODS FOR PRODUCING CIRCULAR RNAS

(71) Applicant: Peking University, Beijing (CN)

(72) Inventors: Wensheng WEI, Beijing (CN); Liang QU, Beijing (CN); Zongyi YI, Beijing (CN); Yong SHEN, Beijing (CN)

(21) Appl. No.: 18/721,131

(22) PCT Filed: Mar. 22, 2022
(86) PCT No.: PCT/CN2022/082224

§ 371 Date: Jun. 17, 2024

(30) Foreign Application Priority Data
Dec. 21, 2021 (WO) PCT/CN2021/140099

(51) Int. Cl. C12P 19/34

(52) U.S. Cl. CPC C12P 19/34

(57) ABSTRACT

The present application provides methods for producing circular RNAs (circRNAs) from a DNA construct encoding a linear RNA precursor, wherein the linear RNA precursor comprises from the 5′-end to the 3′ end: a 3′ catalytic Group I intron fragment, a 3′ exon sequence, an effector RNA sequence, a 5′ exon sequence, and a 5′ catalytic Group I intron fragment, wherein the method comprises an in vitro single-pot reaction. In some embodiments, the single-pot reaction does not comprise supplementing the reagent composition with GTP, a divalent metal ion such as Mg2+, or DNase I prior to circularization of a linear RNA precursor.

### CROSS REFERENCE TO RELATED APPLICATIONS

This application claims priority benefit from International Patent Application No. PCT/CN2021/140099 filed on Dec. 21, 2021, the content of which is incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 165392000941SEQLIST.TXT, date recorded: Mar. 16, 2022, size: 4,809 bytes).

### FIELD

The present application relates to methods for producing circular RNAs (circRNAs).

### BACKGROUND

RNA is increasingly being used as a therapeutic compound or as part of a therapeutic method. This includes use of different types of RNAs for gene silencing, including, for example, siRNA, miRNA, and gRNA. More recently, the development of RNA-based vaccines against SARS-CoV2 has shown the potential for broad application of RNA-based vaccines. Using circRNAs in these RNA-based therapeutics likely presents several advantages over the conventional use of linear RNAs. For example, circRNA is more stable than linear RNA because it is more resistant to enzymatic catalysis. Furthermore, circRNAs do not require nucleotide modifications, while canonical linear RNA agents incorporate nucleotide modifications for improved stability. There is thus a need to efficiently and cost effectively prepare circRNAs.

### BRIEF SUMMARY

The present application provides methods of producing circRNAs from a DNA construct.

One aspect of the present application provides a method of producing a circular RNA from a DNA construct encoding a linear RNA precursor, wherein the linear RNA precursor comprises from the 5′-end to the 3′ end: a 3′ catalytic Group I intron fragment, a 3′ exon sequence, an effector RNA sequence, a 5′ exon sequence, and a 5′ catalytic Group I intron fragment, wherein the method comprises an in vitro single-pot reaction step comprising contacting the DNA construct with a reagent composition comprising an RNA polymerase, adenosine 5′-triphosphate (ATP), uridine 5′-triphosphate (UTP), guanosine 5′-triphosphate (GTP) and cytosine 5′-triphosphate (CTP) under conditions that allow transcription of the DNA construct into the linear RNA precursor and circularization of the linear RNA precursor, wherein the circularization comprises activation of the 3′ catalytic Group I intron fragment and the 5′ catalytic Group I intron fragment to splice the 3′ exon sequence and the 5′ exon sequence from the linear RNA precursor, thereby forming the circular RNA comprising the effector RNA. In some embodiments, the single-pot reaction step does not comprise supplementing the reagent composition with guanosine 5′-triphosphate (GTP) prior to the circularization of the linear RNA precursor.

In some embodiments according to any one of the methods described above, the single-pot reaction step does not comprise supplementing the reagent composition with a divalent metal ion prior to the circularization of the linear RNA precursor. In some embodiments, the divalent metal ion is Mg²⁺.

In some embodiments according to any one of the methods described above, the single-pot reaction step does not comprise incubating the linear RNA precursor at about 55° C.

In some embodiments according to any one of the methods described above, the single-pot reaction step does not comprise DNase I treatment prior to the circularization of the linear RNA precursor.

In some embodiments according to any one of the methods described above, the RNA polymerase is T7 RNA polymerase, a T3 RNA polymerase, or an SP6 RNA polymerase. In some embodiments, the RNA polymerase is T7 RNA polymerase.

In some embodiments according to any one of the methods described above, the reagent composition comprises about 0.01 to about 50 mM of each of ATP, UTP, GTP and CTP. In some embodiments, the reagent composition comprises about 10 mM of each of ATP, UTP, GTP and CTP.

In some embodiments according to any one of the methods described above, the DNA construct is contacted with the reagent composition at about 37° C.

In some embodiments according to any one of the methods described above, the DNA construct is contacted with the reagent composition for at least 20 minutes, such as at least 1 hour, 2 hours, 6 hours, 12 hours, 16 hours, 24 hours or more. In some embodiments, the DNA construct is contacted with the reagent composition for about 20 minutes to about 24 hours.

In some embodiments according to any one of the methods described above, the DNA construct is a plasmid. In some embodiments, the method further comprising linearizing the plasmid. In some embodiments, the plasmid is linearized by reaction enzyme digestion or PCR amplification.

In some embodiments according to any one of the methods described above, the method further comprises isolating the circular RNA. In some embodiments, the circular RNA is isolated by gel purification or high performance liquid chromatography (HPLC).

In some embodiments according to any one of the methods described above, the 3′ catalytic Group I intron and the 5′ catalytic Group I intron are derived from Anabaena Group I intron. In some embodiments, the 3′ catalytic Group I intron comprises the nucleic acid sequence of SEQ ID NO: 1, and the 5′ catalytic Group I intron comprises the nucleic acid sequence of SEQ ID NO: 2.

In some embodiments according to any one of the methods described above, the 3′ exon sequence comprises the nucleic acid sequence of SEQ ID NO: 3, and the 5′ exon sequence comprises the nucleic acid sequence of SEQ ID NO: 4.

In some embodiments according to any one of the methods described above, the linear RNA precursor further comprises a 5′ homology arm sequence flanking the 5′ of the 3′ catalytic Group I intron fragment, and a 3′ homology arm sequence flanking the 3′ of the 5′ catalytic Group I intron fragment, wherein the 5′ homology arm sequence and the 3′ homology arm sequence hybridize with each other. In some embodiments, the 5′ homology arm sequence and the 3′ homology arm sequence are each about 5 to 100 nucleotides in length. In some embodiments, the 5′ homology arm sequence comprises the nucleic acid sequence of SEQ ID NO: 5, and the 3′ homology arm sequence comprises the nucleic acid sequence of SEQ ID NO: 6.

In some embodiments according to any one of the methods described above, the effector RNA sequence comprises a nucleic acid sequence encoding a therapeutic polypeptide. In some embodiments, the therapeutic polypeptide is selected from the group consisting of an antigenic polypeptide, a functional protein, a receptor protein, and a targeting protein. In some embodiments, the effector RNA sequence comprises a Kozak sequence operably linked to the to the nucleic acid sequence encoding the therapeutic polypeptide. In some embodiments, the effector RNA sequence comprises an in-frame 2A peptide coding sequence operably linked to the 3′ end of the nucleic acid sequence encoding the therapeutic polypeptide. In some embodiments, the effector RNA sequence comprises an internal ribosomal entry site (IRES) sequence operably linked to the nucleic acid sequence encoding the therapeutic polypeptide. In some embodiments, the effector RNA sequence comprises an m6A modification motif sequence operably linked to the nucleic acid sequence encoding the therapeutic polypeptide.

In some embodiments according to any one of the methods described above, the effector RNA sequence comprises a nucleic acid sequence comprising a therapeutic RNA.

Another aspect of the present application provides a circular RNA prepared using the method of any one of the methods described above.

### BRIEF DESCRIPTION OF THE DRAWINGS

FIG. 1A shows an exemplary method of generating a circRNA in vitro based on a Group I catalytic intron. A typical Group I catalytic intron comprises, from the 5′ end to the 3′ end: a 5′ exon comprising a 5′ exon sequence recognizable by a 5′ catalytic Group I intron fragment (Exon 1), 5′ catalytic Group I intron fragment, 3′ catalytic Group I intron fragment, and a 3′ exon comprising a 3′ exon sequence recognizable by the 3′ catalytic Group I intron fragment (Exon 2). A linear RNA construct with an insert sequence can be made to allow auto-catalysis of the Group I intron fragments in order to join the two ends of the insert sequence and obtain a circular RNA after self-splicing by the Group I intron. The linear construct comprises, from 5′ to 3′, 3′ catalytic Group I intron fragment, a 3′ exon (Exon 2), an insert sequence, a 5′ exon (Exon 1), and 5′ Group I intron.

FIG. 1B shows a schematic of an exemplary single-pot method of producing a circRNA from a linearized DNA construct by in vitro transcription (“IVT”), in which the IVT conditions allow ribozyme autocatalysis of the Group I catalytic intron. The method is carried out without DNase I treatment or additional GTP treatment. The effector RNA sequence encoded by the linear DNA construct has from the 5′ to the 3′: an m6A modification motif sequence before the start codon, Kozak sequence, signal peptide sequence, a sequence encoding a spike protein, a stop codon (TAA) and a self-splicing 2A peptide.

FIG. 2 shows the results of agarose gel electrophoresis of an exemplary circRNA, circRNA^{RBD}, generated using the indicated conditions, demonstrating that circRNA is produced in a single-pot reaction step as described in Example 1.

### DETAILED DESCRIPTION

The present application provides methods of producing a circRNA using a single-pot in vitro transcription reaction. In some embodiments, the present application provides a method of producing a circRNA without the need to supplement the reagents after in vitro transcription. The methods allow in vitro production of circRNA without additional steps requiring a separate DNase I treatment step or a separate incubation step with GTP and a divalent metal ion (e.g., Mg²⁺).

CircRNAs are typically prepared by in vitro transcription of a DNA template into a linear RNA precursor, followed by DNase I treatment that removes the DNA template, and a separate step to circularize of the linear RNA precursor, for example, by activation of self-splicing of Group I intron fragments in a linear RNA precursor through a GTP treatment step. The known circRNA preparation methods include multiple reaction and purification steps, which are time consuming. The present application is based at least in part on the surprising discovery that the Group I intron fragments in the linear RNA precursors described herein can circularize by self-splicing upon in vitro transcription in a single-pot reaction, without the DNase I or GTP treatment steps. The single-pot methods described herein save time and reagents and is beneficial for RNA-based applications because it reduces potential contaminants that may be immunogenic and/or could lead to RNA degradation.

### I. Definitions

Terms are used herein as generally used in the art, unless otherwise defined as follows.

The term “linear RNA” refers to a RNA molecule having a 5′ end and a 3′ end. A linear RNA may have secondary structures, including helices and loop regions.

The terms “polynucleotide,” “nucleic acid,” “nucleotide sequence,” and “nucleic acid sequence” are used interchangeably. They refer to a polymeric form of nucleotides of any length, either deoxyribonucleotides or ribonucleotides, or analogs thereof.

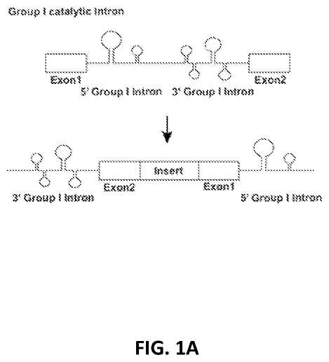

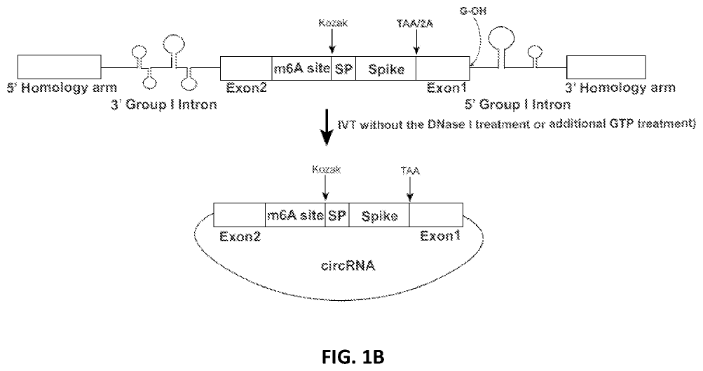

The terms “Group I intron” and “Group I catalytic intron” are used interchangeably to refer to a self-splicing ribozyme that can catalyze its own excision from an RNA precursor. Group I introns comprise two fragments, the 5′ catalytic Group I intron fragment and the 3′ catalytic Group I intron fragment, which retain their folding and catalytic function (i.e., self-splicing activity). In its native environment, the 5′ catalytic Group I intron fragment is flanked at its 5′ end by a 5′ exon, which comprises a 5′ exon sequence that is recognized by the 5′ catalytic Group I intron fragment; and the 3′ catalytic Group I intron fragment is flanked at its 3′ end by a 3′ exon, which comprises a 3′ exon sequence that is recognized by the 3′ catalytic Group I intron fragment. The terms “5′ exon sequence” and “3′ exon sequence” used herein are labeled according to the order of the exons with respect to the Group I intron in its natural environment, e.g., as shown in FIG. 1A.

The term “single-pot reaction” is a term of the art understood by skilled persons and refers to a multi-step reaction or synthesis that takes place within the same reaction vessel and without subsequent separation and/or purification of intermediary compounds.

As used herein, “complementarity” refers to the ability of a nucleic acid to form hydrogen bond(s) with another nucleic acid by traditional Watson-Crick base-pairing. A percent complementarity indicates the percentage of residues in a nucleic acid molecule which can form hydrogen bonds (i.e., Watson-Crick base pairing) with a second nucleic acid (e.g., about 5, 6, 7, 8, 9, 10 out of 10, being about 50%, 60%, 70%, 80%, 90%, and 100% complementary respectively). “Perfectly complementary” means that all the contiguous residues of a nucleic acid sequence form hydrogen bonds with the same number of contiguous residues in a second nucleic acid sequence. “Substantially complementary” as used herein refers to a degree of complementarity that is at least about any one of 70%, 75%, 80%, 85%, 90%, 95%, 97%, 98%, 99%, or 100% over a region of about 40, 50, 60, 70, 80, 100, 150, 200, 250 or more nucleotides, or refers to two nucleic acids that hybridize under stringent conditions.

As used herein the term “wild type” is a term of the art understood by skilled persons and means the typical form of an organism, strain, gene or characteristic as it occurs in nature as distinguished from mutant or variant forms.

The present disclosure provides several types of compositions that are polynucleotide or polypeptide based, including variants and derivatives. These include, for example, substitutional, insertional, deletion and covalent variants and derivatives. The term “derivative” is synonymous with the term “variant” and generally refers to a molecule that has been modified and/or changed in any way relative to a reference molecule or a starting molecule.

As such, polynucleotides encoding peptides or polypeptides containing substitutions, insertions and/or additions, deletions and covalent modifications with respect to reference sequences, in particular, the polypeptide sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal residues or N-terminal residues) alternatively may be deleted depending on the use of the sequence, as for example, expression of the sequence as part of a larger sequence that is soluble, or linked to a solid support.

The term “identity” refers to the overall relatedness between polymeric molecules, for example, between polynucleotide molecules (e.g. DNA molecules and/or RNA molecules) and/or between polypeptide molecules. Calculation of the percent identity of two polynucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleic acid sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleic acid sequences can be determined using the algorithm of Meyers and Miller (CABIOS, 1989, 4:11-17), which has been incorporated into the ALIGN program (version 2.0) using a PAM 120 weight residue table, a gap length penalty of 12 and a gap penalty of 4. The percent identity between two nucleic acid sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

“Percent (%) amino acid sequence identity” with respect to the polypeptide sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32(5):1792-1797, 2004; Edgar, R. C., BMC Bioinformatics 5(1):113, 2004, each of which are incorporated herein by reference in their entirety for all purposes).

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat disease of type X means the method is used to treat disease of types other than X.

The term “about X-Y” used herein has the same meaning as “about X to about Y.”

As used herein and in the appended claims, the singular forms “a,” “an,” or “the” include plural referents unless the context clearly dictates otherwise.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

II. Methods of Producing circRNAs

The present application provides methods for producing circRNAs comprising a single-pot reaction step and circRNAs prepared using the described methods.

In some embodiments, there is provided a method for producing a circular RNA from a DNA construct encoding a linear RNA precursor, wherein the linear RNA precursor comprises from the 5′-end to the 3′ end: a 3′ catalytic Group I intron fragment, a 3′ exon sequence, an effector RNA sequence, a 5′ exon sequence, and a 5′ catalytic Group I intron fragment, wherein the method comprises an in vitro single-pot reaction step comprising contacting the DNA construct with a reagent composition comprising an RNA polymerase, adenosine 5′-triphosphate (ATP), uridine 5′-triphosphate (UTP), guanosine 5′-triphosphate (GTP) and cytosine 5′-triphosphate (CTP) under conditions that allow transcription of the DNA construct into the linear RNA precursor and circularization of the linear RNA precursor, wherein the circularization comprises activation of the 3′ catalytic Group I intron fragment and the 5′ catalytic Group I intron fragment to splice the 3′ exon sequence and the 5′ exon sequence from the linear RNA precursor, thereby forming the circular RNA comprising the effector RNA.

In some embodiments, the single-pot reaction step does not comprise supplementing the reagent composition with new and/or additional reagents. In some embodiments, the single-pot reaction step does not comprise removing one or more starting materials, such as unreacted DNA construct, NTPs, RNA polymerase, etc., from the reaction mixture before circularization of the linear RNA precursor. In some embodiments, the single-pot reaction step does not comprise isolating or purifying the linear RNA precursor prior to circularization of the linear RNA precursor.

In some embodiments, the single-pot reaction step does not comprise a GTP treatment step. GTP treatment refers to a reaction in which a linear RNA precursor containing Group I intron fragments is contacted with one or more reagents to activate self-splicing of the Group I intron fragments. As shown in FIG. 1B, during self-splicing, the 3′ hydroxyl group of a guanosine nucleotide engages in a transesterification reaction at the 5′ splice site. The 5′ intron half is excised, and the freed hydroxyl group at the end of the intermediate engages in a second transesterification at the 3′ splice site, resulting in circularization of the intervening region and excision of the 3′ intron. A GTP treatment step comprises contacting the linear RNA precursor with GTP (e.g., at a final concentration of 2 mM). A GTP treatment step may further comprise contacting the linear RNA precursor with a divalent metal ion, such as Mg²⁺. In some embodiments, a GTP treatment step comprises contacting the linear RNA precursor with GTP and Mg²⁺ at about 55° C. for about 8 minutes.

In some embodiments, the single-pot reaction step does not comprise supplementing the reagent composition with one or more nucleoside triphosphates prior to the circularization of the linear RNA precursor. In some embodiments, the single-pot reaction step does not comprise supplementing the reagent composition with GTP. In some embodiments, the NTP mixture for in vitro transcription is sufficient to allow activation of self-splicing of the Group I intron fragments in the linear RNA precursor.

In some embodiments, the single-pot reaction step does not comprise supplementing the reaction composition with a divalent metal ion prior to circularization of the linear RNA precursor. In some embodiments, the divalent metal ion is selected from the group consisting of Mg²⁺, Mn²⁺, Ca²⁺, Co²⁺, Be²⁺, Cu²⁺, Fe²⁺, Zn²⁺, Sr²⁺, Ba²⁺, Al²⁺, and Cd²⁺. In some embodiments, the single-pot reaction step does not comprise supplementing the reaction composition with Mg²⁺.

In some embodiments, the single-pot reaction step does not comprise supplementing the reagent composition with GTP or a divalent metal ion such as Mg²⁺.

In some embodiments, the single-pot reaction step does not comprise incubating the linear RNA precursor at about at least 40, 45, 50, 55, 60, 65, or 70° C. In some embodiments, the single-pot reaction step does not comprise incubating the linear RNA precursor at about any one of 40-50, 50-60, 60-70, 40-70 or 50-60° C. In some embodiments, the single-pot reaction step does not comprise incubating the linear RNA precursor at about 55° C.

In known methods of producing circularized RNA, the in vitro transcription product from the DNA construct is subject to a number of treatment and/or reaction steps, including DNase I treatment and isolation of the linear RNA precursor prior to circularization by activation of the Group I intron fragments. DNase I treatment removes DNA construct from the reaction mixture after completion of in vitro transcription. A typical DNase I treatment step is described in Example 1, which includes treating in vitro transcribed circRNA precursors with DNase I.

In some embodiments, the single-pot reaction step does not comprise DNase treatment prior to circularization of the linear RNA precursor. In some embodiments, the DNase is DNase I or DNase II. In some embodiments, the DNase is a micrococcal nuclease. In some embodiments, the DNase is a restriction enzyme. In some embodiments, the single-pot reaction step does not comprise DNase I treatment prior to circularization of the linear RNA precursor.

In some embodiments, the single-pot reaction step does not comprise contacting the product of in vitro transcription with a DNase I at 37° C. for about 20 minutes. In some embodiments, the single-pot reaction step does not comprise isolating and/or purifying the linear RNA precursor from the in vitro transcription reaction.

In some embodiments, the RNA polymerase is a T7 RNA polymerase, a T3 RNA polymerase, a SP6 RNA polymerase, or a derivative thereof. In some embodiments, the in vitro transcription is driven by a T7 promoter in the DNA construct, and the RNA polymerase is a T7 RNA polymerase. In some embodiments, the in vitro transcription is driven by a T3 phage promoter in the DNA, and the RNA polymerase is a T3 RNA polymerase. In some embodiments, the in vitro transcription is driven by an SP6 promoter in the DNA construct, and the RNA polymerase is a SP6 RNA polymerase.

In some embodiments, the reagent composition comprises an NTP mixture. In some embodiments, the NTP mixture comprises ATP, UTP, GTP and CTP. In some embodiments, the NTP mixture comprises one or more modified nucleoside 5′ triphosphate. In some embodiments, the NTP mixture does not comprise a modified nucleoside 5′ triphosphate. In some embodiments, the reagent composition comprises an equal concentration for each of ATP, UTP, GTP, and CTP. In some embodiments, the reagent composition comprises an equal concentration for at least two of ATP, UTP, GTP, or CTP. In some embodiments, the reagent composition comprises a different concentration for each of ATP, UTP, GTP, and CTP. In some embodiments, the concentration of GTP in the reagent composition is higher than one or more of the concentrations of ATP, UTP and GTP. In some embodiments, the concentration of a nucleoside 5′ triphosphate (e.g., ATP, UTP, GTP or CTP) in the reagent composition is at least about 0.01, 0.05, 0.1, 0.5, 1, 2, 3, 4, 5, 6, 7, 8, 9, 10, 15, 20, 30, 40, 50 mM, or higher. In some embodiments, the concentration of a nucleoside 5′ triphosphate (e.g., ATP, UTP, GTP or CTP) in the reagent composition is no more than about any one of 50, 40, 30, 20, 15, 10, 9, 8, 7, 6, 5, 4, 3, 2, 1, 0.05, 0.01 mM or less. In some embodiments, the concentration of a nucleoside 5′ triphosphate (e.g., ATP, UTP, GTP or CTP) in the reagent composition is about any one of 0.01-0.05, 0.05-0.1, 0.1-0.5, 0.5-1, 1-2, 2-3, 3-4, 4-5, 5-6, 6-7, 7-8, 8-9, 9-10, 10-15, 15-20, 20-30, 30-40, 40-50, 0.01-10, 0.01-50, 0.1-10, 0.1-50, 10-50, 5-20, 20-40 or 5-25 mM. In some embodiments, the concentration of each of ATP, GTP, CTP and UTP in the reaction composition is about 10 mM. In some embodiments, the concentration of GTP is about 7.5 mM. In some embodiments, the concentration of each of ATP, UTP and CTP is the same, and the concentration of GTP is higher than the concentration of each of ATP, UTP and CTP. In some embodiments, the concentration of GTP is about at least any one of 1.1, 1.2, 1.25, 1.3, 1.4, 1.5, 1.6, 1.7, 1.75, 1.8, 1.9, 2, 2.5, 3, 3.5, 4 times or more than the concentration of ATP, UTP or CTP.

In some embodiments, the DNA construct is contacted with the reagent composition at about at least any one of 37, 38, 39, 40, 41, 42, 43, 44, 45, 46, 47, 48, 49, 50, 51, 52, 53, 54, 55, or 56° C. In some embodiments, the DNA construct is contacted with the reagent composition at no more than about any one of 56, 54, 53, 52, 51, 50, 49, 48, 47, 46, 45, 44, 43, 42, 41, 40, 39, 38, or 37° C. In some embodiments, the DNA construct is contacted with the reagent composition at about any one of 37-38, 38-40, 40-42, 42-44, 44-46, 46-48, 48-50, 50-52, 52-54, 54-56, 37-45, 45-56, or 40-50° C. In some embodiments, the DNA construct is contacted with the reagent composition at about 37° C.

In some embodiments, the DNA construct is contacted with the reagent composition for at least about any one of 5, 10, 20, 30, or 40 minutes, or 1, 2, 3, 4, 5, 6, 7, 8, 9, 10, 11, 12, 13, 14, 15, 16, 17, 18, 19, 20, 21, 22, 23, or 24 hours. In some embodiments, the DNA construct is contacted with the reagent composition for no more than about any one of 24, 23, 22, 21, 20, 19, 18, 17, 16, 15, 14, 13, 12, 11, 10, 9, 8, 7, 6, 5, 4, 3, 2, or 1 hour(s), or 40, 30, 20, 10, or 5 minutes. In some embodiments, the DNA construct is contacted with the reagent composition for about any one of 1-2, 2-4, 4-6, 6-8, 8-10, 10-12, 12-16, 16-18, 18-20, 20-22, 22-24, 1-6, 6-12, 12-18, 18-24, 1-12, 12-24, or 6-16 hours, or 20 minutes to 1 hour, or 20 minutes to 2 hours, or 20 minutes to 16 hours, or 20 minutes to 24 hours. In some embodiments, the DNA construct is contacted with the reagent composition for about 16 hours. In some embodiments, the DNA construct is contacted with the reagent composition for about 20 minutes to about 24 hours.

In some embodiments, the method comprises one or more additional steps for obtaining the DNA construct and/or isolating the circular RNA.

In some embodiments, the method comprises producing the circular RNA from a DNA construct encoding a linear RNA precursor. In some embodiments, the DNA construct is a plasmid. In some embodiments, the method comprises linearizing the plasmid. In some embodiments, the plasmid is linearized by restriction enzyme digestion. In some embodiments, the plasmid is linearized by PCR amplification.

In some embodiments, the method comprises treating the product of the single-pot reaction step with RNase R to digest the linear RNA precursor molecules that are not circularized. In some embodiments, the method does not comprise treating the product of the single-pot reaction step with RNase R to digest the linear RNA precursor molecules that are not circularized.

In some embodiments, the method further comprises a step of purifying the circularized RNA product. In non-limiting examples, the circRNA is purified by gel-purification or by high-performance liquid chromatography (HPLC). In some embodiments, agarose gel electrophoresis allows for simple and effective separation of circular splicing products from linear precursor molecules, nicked circles, splicing intermediates, and excised introns. In some embodiments, the method comprises purifying the circular dRNA by chromatography, such as HPLC. In some embodiments, the purified circular dRNA can be stored at −80° C.

### Linear RNA Precursor

In some embodiments, the present application provides a linear RNA capable of forming the circRNA of any one of the embodiments described herein. In some embodiments, the present application provides a linear RNA capable of forming the circRNA of any one of the embodiments described herein, wherein the linear RNA can be circularized by autocatalysis of a Group I intron. In some embodiments, the Group I intron comprises a 5′ catalytic Group I intron fragment and a 3′ catalytic Group I intron fragment. In some embodiments, the linear RNA comprises a 3′ catalytic Group I intron fragment (such as the sequence set forth in SEQ ID NO: 1) flanking the 5′ end of a 3′ exon sequence recognizable by the 3′ catalytic Group I intron fragment (such as the sequence set forth in SEQ ID NO: 3), and the 5′ catalytic Group I intron fragment (such as the sequence set forth in SEQ ID NO: 2) flanking the 3′ end of a 5′ exon sequence recognizable by the 5′ catalytic Group I intron fragment (such as the sequence set forth in SEQ ID NO: 4).

The 3′ catalytic Group I intron, 5′ catalytic Group I intron, 3′ exon and 5′ exon are derived from a Group I intron. Any Group I intron known in the art could be used to generate circRNA via self-splicing. Examples of Group I introns useful for the methods of this application are described in Puttaraju, M. & Been, M., Nucleic Acids Res. 20, 5357-5364 (1992); Ford, E. & Ares, M., Proc. Natl Acad. Sci. 91, 3117-3121 (1994); Vicens, Q., Paukstelis, P. J., Westhof, E., Lambowitz, A. M. & Cech, T. R., RNA 14, 2013-2029 (2008), which are incorporated herein by reference.

In some embodiments, the 3′ catalytic Group I intron and the 5′ catalytic Group I intron are derived from a bacterial phage Group I intron, such as a Group I intron of a T4 phage. In some embodiments, the 3′ catalytic Group I intron and the 5′ catalytic Group I intron are derived from a bacterial Group I intron. In some embodiments, the 3′ catalytic Group I intron and the 5′ catalytic Group I intron are derived from a cyanobacteria Group I intron. In some embodiments, the 3′ catalytic Group I intron and the 5′ catalytic Group I intron are derived from the Anabaena Group I intron. In some embodiments, the 3′ catalytic Group I intron comprises the nucleic acid sequence of SEQ ID NO: 1, and the 5′ catalytic Group I intron comprises the nucleic acid sequence of SEQ ID NO: 2.

In some embodiments, the 3′ exon sequence and the 5′ exon sequence are derived from the Anabaena Group I Intron. In some embodiments, the 3′ exon sequences comprises the nucleic acid sequence of SEQ ID NO: 3, and the 5′ exon sequence comprises the nucleic acid sequence of SEQ ID NO: 4.

In some embodiments, the linear RNA precursor further comprises a 5′ homology arm sequence flanking the 5′ of the 3′ catalytic Group I intron fragment, and a 3′ homology arm sequence flanking the 3′ of the 5′ catalytic Group I intron fragment, wherein the 5′ homology arm sequence and the 3′ homology arm sequence hybridize with each other. In some embodiments, the 5′ homology arm sequence and the 3′ homology arm sequence are each about 5, 10, 15, 20, 25, 30, 35, 40, 45, 50, 55, 60, 65, 70, 75, 80, 85, 90, 95, or 100 nucleotides in length. In some embodiments, the 5′ homology arm sequences comprises the nucleic acid sequence of SEQ ID NO: 5, and the 3′ homology arm sequence comprises the nucleic acid sequence of SEQ ID NO: 6.

The linear RNA precursors and circRNAs described herein comprise an effector RNA sequence, which may be a coding RNA sequence or a non-coding RNA sequence. Exemplary non-coding RNAs include, but are not limited to, guide RNAs (gRNA, including single guide RNA or sgRNA), a deaminase-recruiting RNA (dRNA), a small RNA (such as a microRNA, a short hairpin RNA, or a small interfering RNA), or a long intervening non-coding RNA (lincRNA).

In some embodiments, the effector RNA sequence is at least about 50 nt long, such as at least about any one of 100, 150, 200, 300, 600, 900, 1200, 1500, 2000, 3000, 4000, 5000, or more nt long. In some embodiments, the effector RNA sequence is no more than about any one of 5000, 4000, 3000, 2000, 1500, 1200, 900, 600, 300, 200, 150, or 100 nt long. In some embodiments, the effector RNA sequence is about any one of 50-100, 100-500, 500-1000, 1000-2000, 2000-5000, 50-5000, 100-5000, 100-3000, 500-5000, 500-2500, 2500-5000, or 1000-5000 nt long.

In some embodiments, the effector RNA sequence is a coding RNA sequence, which encode any polypeptide of interest. In some embodiments, the polypeptide is at least about 15 amino acids long, such as at least about any one of 20, 50, 100, 200, 300, 400, 500, 600, 700, 800, 900, 1000 or more amino acids long. In some embodiments, the polypeptide is no more than about any one of 1000, 900, 800, 700, 600, 500, 400, 300, 200, 100, 50, or 20 amino acids long. In some embodiments, the polypeptide is about any one of 20-50, 50-100, 20-200, 20-500, 20-1000, 50-500, 50-1000, 100-500, 100-1000, 200-1000 or 500-1000 amino acids long.

In some embodiments, the effector RNA sequence comprises a nucleic acid sequence encoding a therapeutic polypeptide. In some embodiments, the therapeutic polypeptide is selected from the group consisting of an antigenic polypeptide, a functional protein, a receptor protein, and a targeting protein.

In some embodiments, the coding RNA sequence encodes an antigenic polypeptide. A circRNA vaccine may be prepared using a linear RNA comprising a coding RNA sequence encoding an antigenic polypeptide. An antigenic polypeptide comprises at least one epitope recognizable by a T cell receptor (TCR). In some embodiments, the antigenic polypeptide is a full-length protein or a fragment thereof, or an antigenic fusion protein that can trigger an immune response in a subject. In some embodiments, the antigenic polypeptide is a short peptide of no more than 100 amino acids long. The antigenic polypeptide can be a naturally derived peptide fragment from a protein antigen containing one or more epitopes, or an artificially designed peptide with one or more natural epitope sequences, wherein a peptide linker may optionally be placed in between adjacent epitope sequences. In some embodiments, the antigenic polypeptide comprises a single epitope of an antigenic protein. In some embodiments, the antigenic polypeptide comprises about any one of 1, 2, 3, 4, 5, 10 or more epitopes from a single antigenic protein. In some embodiments, the antigenic polypeptide comprises epitopes from a plurality (e.g., 2, 3, 4, 5, 10 or more) of different antigenic proteins. In some embodiments, the antigenic polypeptide comprises a Major Histocompatibility Complex (MHC) class I-restricted epitope. In some embodiments, the antigenic polypeptide comprises a MHC class II-restricted epitope. In some embodiments, the antigenic polypeptide comprises both MHC class I-restricted and MHC class II-restricted epitopes.

In some embodiments, the antigenic polypeptide is an antigenic protein or fragment thereof or a variant thereof from a pathogenic agent, such as a bacterium or a virus. In some embodiments, the antigenic polypeptide is an antigenic protein or fragment of a coronavirus, such as SARS-CoV2, including variants thereof. In some embodiments, the antigenic polypeptide comprises a Spike (S) protein or a fragment thereof or a variant thereof of a coronavirus, such as SARS-CoV, MERS-COV, or SARS-CoV-2. CircRNA vaccines have been described, for example, in PCT/CN2021/074998, which is incorporated herein by reference in its entirety. The linear RNAs and constructs described herein may be used to prepare any one of the known circRNA vaccines in the art.

In some embodiments, the antigenic polypeptide is an antigenic protein or fragment thereof or a variant thereof of a self-antigen, such as an antigen involved in a disease or condition. In some embodiments, the antigenic polypeptide is a tumor antigen peptide. Tumor antigen peptide sequences are known in the art and can be found at public databases, such as the Cancer Antigenic Peptide Database (van der Bruggen P et al. (2013) “Peptide database: T cell-defined tumor antigens.” Cancer Immunity. URL: caped.icp.ucl.ac.be). The coding RNA sequence in the linear RNA or circRNA described herein may encode any of the known tumor antigen peptides or combinations thereof. In some embodiments, the antigenic polypeptide comprises an epitope of a tumor associated antigen (TAA). In some embodiments, the antigenic polypeptide comprises an epitope of a tumor specific antigen. In some embodiments, the antigenic polypeptide comprises an epitope of a neoantigen, i.e., newly acquired and expressed antigens present in tumor cells of an individual.

In some embodiments, the amino acid sequences of one or more epitope peptides are predicted based on the sequence of the antigen protein (including neoantigens) using a bioinformatics tool for T cell epitope prediction. Exemplary bioinformatics tools for T cell epitope prediction are known in the art, for example, see Yang X. and Yu X. (2009) “An introduction to epitope prediction methods and software” Rev. Med. Virol. 19(2): 77-96. In some embodiments, the sequence of the antigen protein is known in the art or available in public databases. In some embodiments, the sequence of the antigen protein (including neoantigens) is determined by sequencing a sample (such as a tumor sample) of the individual being treated.

In some embodiments, the antigenic polypeptide comprises a multimerization domain, such as a dimerization domain, a trimerization domain, or a domain that mediates formation of higher order multimers. In some embodiments, the multimerization domain is a trimerization domain. In non-limiting examples, the multimerization domain comprises a C-terminal Foldon (Fd) domain of a T4 fibritin protein, wherein the C-terminal Foldon domain is the domain that mediates trimerization of the T4 fibritin protein. In another example, the multimerization domain comprises a GCN4-based isoleucine zipper (IZ) domain based on the trimerization domain of the GCN4 transcriptional activator from Saccharomyces cerevisiae. In some embodiments, the GCN4 IZ domain or T4 fibritin Fd domain can be modified to reduce their immunogenicity according to known techniques in the art. For example, the GCN4 IZ domain can be modified with N-linked glycosylation sites to reduce its immunogenicity (Sliepen et al. Immunosilencing a Highly Immunogenic Protein Trimerization Domain. The Journal of Biol. Chem. Vol. 290, No. 12, pp. 7436-7442).

In some embodiments, the antigenic polypeptide further comprises an immunogenic carrier protein. In some embodiments, the antigenic polypeptide comprises an epitope peptide conjugated to an immunogenic carrier protein. Exemplary immunogenic carrier proteins include, but are not limited to, tetanus toxoid (TT), diphtheria toxoid (DT), modified cross-reacting material of diphtheria toxin (CRM197), meningococcal outer membrane protein complex (OMPC), and Hemophilus influenzae protein D (HiD).

In some embodiments, the coding RNA sequence encodes a targeting protein. In some embodiments, the targeting protein is an antibody or an antigen-binding fragment thereof.

In some embodiments, the coding RNA sequence encodes an antibody. In some embodiments, the therapeutic polypeptide is a neutralizing antibody, i.e., an antibody that blocks an interaction between a protein and its binding partner. In some embodiments, the antibody inhibits activity of a protein, e.g., by blocking binding of the protein to a binding partner. In some embodiments, the targeting protein is a therapeutic antibody. In some embodiments, the antibody is a checkpoint inhibitor, e.g., an antibody inhibitor of CTLA-4, PD-1, or PD-L1. In some embodiments, the antibody specifically binds a cell surface antigen, such as a tumor antigen. Exemplary tumor antigens include, but are not limited to, glioma-associated antigen, carcinoembryonic antigen (CEA), β-human chorionic gonadotropin, alphafetoprotein (AFP), lectin-reactive AFP, thyroglobulin, RAGE-1, MN-CAIX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, HER2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin. In some embodiments, the antibody specifically binds a target antigen on a pathogenic agent, such as a bacterium or a virus.

The antibody can be an antigen-binding fragment of an antibody, e.g., a portion or fragment of an intact or complete antibody having fewer amino acid residues than the intact or complete antibody, which is capable of binding to an antigen or competing with the intact antibody (i.e., the intact antibody from which the antigen-binding fragment is derived) for binding to an antigen. Antigen-binding fragments can be prepared by recombinant DNA techniques, or by enzymatic or chemical cleavage of intact antibodies. Antigen binding fragments include, but are not limited to, Fab′, F (ab′)2Fv, single chain Fv (scFv), single chain Fab, diabody (diabody), single domain antibody (sdAb, nanobody), camel Ig, Ig NAR, F (ab)′3Fragment, bis-scFv, (scFv)2Minibodies, diabodies, triabodies, tetradiabodies, disulfide stabilized Fv proteins (“dsFv”). In some embodiments, the neutralizing antibody can be a genetically engineered antibody, such as a chimeric antibody (e.g., humanized murine antibodies), heteroconjugate antibody (e.g., bispecific antibodies), or antigen-binding fragments thereof.

In some embodiments, the antibody is a neutralizing antibody that binds to a viral protein. In some embodiments, the antibody is a neutralizing antibody that binds to a receptor for a viral protein. In some embodiments, the antibody binds to a receptor that is required for viral entry into a cell (e.g., an ACE2 receptor). In some embodiments, the antibody is a neutralizing antibody (nAb) that binds to the S protein of a coronavirus and prevents or reduces its ability to infect cells. In some embodiments, the coronavirus is SARS-CoV-2. In some embodiments, the nAb binds to a S protein comprising one or more mutations. In some embodiments, the nAb binds to a S protein or fragment thereof that comprises at least one point mutation in the S2 region, for example, a K986P, V987P, F817P, A892P, A899P or A942P mutation or combinations thereof. In some embodiments, the nAb binds to a S protein or fragment thereof that comprises at least one point mutation selected from A222V, G339D, S371L, S373P, S375F, E406W, K417N, K417T, N439K, N440K, G446S, L455N, S477N, T478K, E484A, E484K, Q493F, G496S, Q498R, N501Y, Y505H, T547K, A570D, D614G, H655Y, P681H, A701V, T7161, N764K, D796Y, N856K, Q954H, N969K, L981F, S982A, or combinations thereof. In some embodiments the nAb is a monoclonal antibody (mAb), a functional antigen-binding fragment (Fab), a single-chain variable region fragment (scFv), or a single-domain antibody (a VHH or nanobody).

Exemplary nAbs for binding and neutralization of the S protein of SARS-CoV-2 have been described, for example, in Barnes, C.O. et al. SARS-CoV-2 neutralizing antibody structures inform therapeutic strategies. Nature 588, 682-687 (2020), and Chinese Patent Application No. CN111690058A, the contents of which are herein incorporated by reference in their entirety.

In some embodiments, the coding RNA sequence encodes a targeting protein that is not an antibody. Examples of non-antibody-based targeting proteins include, but are not limited to, a lipocalin, an anticalin (artificial antibody mimetic proteins that are derived from human lipocalins), “T-body”, a peptide (e.g., a BICYCLE™ peptide), an affibody (antibody mimetics composed of alpha helices, e.g. an three-helix bundle), a peptibody (peptide-Fc fusion), a DARPin (designed ankyrin repeat proteins, engineered antibody mimetic proteins consisting repeat motifs), an affimer, an avimer, a knottin (a protein structural motif containing 3 disulfide bridges), a monobody, an affinity clamp, an ectodomain, a receptor ectodomain, a receptor, a cytokine, a ligand, an immunocytokine, and a centryin. See, for example, Vazquez-Lombardi, Rodrigo, et al. Drug discovery today 20.10 (2015): 1271-1283.

In some embodiments, the coding RNA sequence encodes a soluble receptor. Soluble receptors (sometimes referred to as soluble receptor decoys or “traps”) can comprise all or a portion of the extracellular domain of a receptor protein. In some embodiments, a nucleotide sequence encoding all or a portion of the extracellular domain of a receptor protein is operably linked to a signal peptide for secretion from cells.

In some embodiments, the soluble receptor comprises an extracellular domain of a naturally occurring receptor. In some embodiments, the soluble receptor variant comprises an engineered variant of an extracellular domain of a naturally occurring receptor, such as a variant comprising one or more mutations in the extracellular domain. In some embodiments, the soluble receptor comprises one or more mutations that increase the affinity of the soluble receptor for its ligand compared to the affinity of the naturally occurring receptor for its ligand.

In some embodiments, the soluble receptor is a fusion protein comprising one or more additional protein domains operably linked to the extracellular domain of the receptor or a variant thereof. In some embodiments, the soluble receptor comprises an Fc domain of an immunoglobulin (Ig), e.g., a human immunoglobulin. In some embodiments, the soluble receptor comprises an Fc domain of a human IgG1.

In some embodiments, the soluble receptor comprises the extracellular domain of a signaling receptor, and the soluble receptor can reduce or inhibit activity of the signaling pathway by blocking binding between the endogenous receptor and its ligand.

In some embodiments, the soluble receptor is a receptor that binds to a viral protein and/or that mediates viral entry. In some embodiments, soluble receptor is a soluble ACE2 receptor. In some embodiments, the therapeutic polypeptide is a soluble ACE2 receptor variant capable of binding to an S protein of a coronavirus. In some embodiments, the soluble ACE2 receptor variant binds to the receptor binding domain (RBD) of the S protein. In some embodiments, the ACE2 receptor variant is enzymatically active. In other embodiments, the ACE2 receptor variant is enzymatically inactive. In some embodiments, the soluble ACE2 receptor variant comprises the soluble extracellular domain of wild-type (WT) human recombinant ACE2 (APN01). In some embodiments, the soluble ACE2 receptor variant comprises one or more mutations in the extracellular domain of human ACE2. In some embodiments, the soluble ACE2 receptor variant is engineered via affinity maturation to have increased binding affinity to the RBD of the S protein. Soluble ACE2 receptor variants have been described, for example in Haschke M et al., Clin Pharmacokinet. 2013 September; 52(9):783-92; Glasgow A et al., Proceedings of the National Academy of Sciences November 2020, 117 (45) 28046-28055; and Higuchi Y. et al., bioRxiv 2020. 09.16.299891, the contents of which are herein incorporated by reference in their entirety. In some embodiments, the soluble ACE2 receptor variant is a fusion protein, e.g., a fusion of the extracellular ACE2 receptor domain to the Fc region of the human IgG1.

In some embodiments, the coding RNA sequence encodes a functional protein. In some embodiments, the coding RNA sequence is capable of being expressed by target cells (e.g., human or mouse cells) for the production (and in certain instances, the secretion) of a functional enzyme or protein as disclosed, for example, in International Application No. PCT/US2010/058457 and WO2020237227, the contents of which are herein incorporated by reference in their entirety. In some embodiments, the therapeutic polypeptide can be engineered for secretion by operably linking a signal peptide to the amino terminus of the therapeutic polypeptide. For example, in some embodiments, upon the expression of one or more therapeutic polynucleotides by target cells, the production of a functional enzyme or protein in which a subject is deficient (e.g., a urea cycle enzyme or an enzyme associated with a lysosomal storage disorder) may be observed.

In some embodiments, the coding RNA sequence encodes a protein such as IDUA, OTC, FAH, miniDMD, DMD, p53, PTEN, COL3A1, BMPR2, AHI1, FANCC, MYBPC3, ILRG2, or ARG1, wherein deficiency of the functional protein is associated with a disease or disorder. In some embodiments, the coding RNA sequence a protein (e.g., a lysosomal enzyme) wherein deficiency of the protein is associated with a lysosomal storage disorder.

In some embodiments, the coding RNA sequence encodes a protein (e.g., an enzyme), wherein deficiency of the protein is associated with a metabolic disorder. In some embodiments, the therapeutic polypeptide comprises a urea cycle enzyme (e.g., ARG1).

In some embodiments, the coding RNA sequence encodes a protein (e.g., p53 or PTEN), wherein deficiency of the protein is associated with a cancer. In some embodiments, the therapeutic polypeptide comprises a tumor suppressor.

In some embodiments, the coding RNA sequence encodes a reporter protein, such as a fluorescent protein. Fluorescent proteins are well known to those skilled in the art, and include but are not limited to, green fluorescent proteins (GFPs), enhanced green fluorescent proteins (EGFPs), red fluorescent proteins (RFPs), and blue fluorescent proteins (BFPs).

In some embodiments, the coding RNA sequence encodes two or more polypeptides, such as two or more therapeutic polypeptides. In some embodiments, the coding RNA sequence encodes a therapeutic polypeptide and a reporter protein.

In some embodiments, the various domains or fragments in the polypeptide encoded by the coding RNA sequence may be fused to each other via a peptide linker. Flexible peptide linkers such as glycine linkers, glycine-serine linkers, and linkers containing other amino acids are known in the art (for example, suitable peptide linkers are described by Chen et al. in Fusion Protein Linkers: Property, Design and Functionality. Adv. Drug Deli Rev. 2013 October 15; 65(10): 1357-1369). Peptide linkers can also be designed by computation methods. The peptide linker can be of any length from 1 to 10, 10 to 20, 20 to 30, 30 to 40, 40 to 50, or greater than 50 amino acids.

In some embodiments, the coding RNA sequence is codon-optimized. A codon optimized sequence may be one in which codons in a polynucleotide encoding a polypeptide have been substituted in order to increase the expression, stability and/or activity of the polypeptide. Factors that influence codon optimization include, but are not limited to one or more of: (i) variation of codon biases between two or more organisms or genes or synthetically constructed bias tables, (ii) variation in the degree of codon bias within an organism, gene, or set of genes, (iii) systematic variation of codons including context, (iv) variation of codons according to their decoding tRNAs, (v) variation of codons according to GC %, either overall or in one position of the triplet, (vi) variation in degree of similarity to a reference sequence for example a naturally occurring sequence, (vii) variation in the codon frequency cutoff, (viii) structural properties of mRNAs transcribed from the DNA sequence, (ix) prior knowledge about the function of the DNA sequences upon which design of the codon substitution set is to be based, and/or (x) systematic variation of codon sets for each amino acid. In some embodiments, a codon optimized polynucleotide may minimize ribozyme collisions and/or limit structural interference between the expression sequence and the IRES.

In some embodiments, the coding RNA sequence may encode or be operably linked to one or more additional elements that facilitate translation of the coding RNA sequence into a functional polypeptide. In some embodiments, the one or more additional elements are useful for monitoring translation of the coding RNA sequence.

In some embodiments, the coding RNA sequence encodes a polypeptide comprising a signal peptide (SP). In non-limiting examples, the signal peptide is the signal sequence and propeptide from human tissue plasminogen activator (tPA), the signal sequence from human IgE Immunoglobulin, or the signal peptide sequence of MHC I. In some embodiments, the signal peptide can facilitate secretion of the polypeptide encoded by the coding RNA sequence.

In some embodiments, the 3′ end of the coding RNA sequence is operably linked to an in-frame 2A peptide coding sequence. In some embodiments, the coding RNA sequence does not comprise a stop codon at the 3′ end. In some embodiments, the in-frame 2A peptide coding sequence replaces the stop codon. In some embodiments, the coding RNA sequence contains no stop codon and the number of nucleotides composing the coding RNA is a multiple of three. In some embodiments, the coding RNA sequence having no stop codon and the number of nucleotides composing the RNA being a multiple of three allow for rolling circle translation of the circRNA prepared using the linear RNA precursor. In some embodiments, the 2A peptide coding sequence allows for rolling circle translation of the circRNA prepared using the linear RNA precursor. In some embodiments, the 2A peptide allows cleavage of a polypeptide generated by rolling circle translation into monomeric polypeptide sequences. In non-limiting examples, the 2A peptide coding sequence encodes a P2A or T2A peptide, such as the sequence set forth in SEQ ID NO: 9 or 12.

In some embodiments, the coding RNA sequence comprises a nucleotide sequence encoding an affinity or identification tag. Exemplary tags include, but are not limited to, His tag, FLAGtag, SUMO tag, GST tag, and MBP tag.

In some embodiments, the 5′ end of the coding RNA sequence is operably linked to a Kozak sequence. In some embodiments, the Kozak sequence functions as a protein translation initiation site. In some embodiments, the linear RNA comprises from the 5′ end to the 3′ end: a first portion of a RNA element (e.g., IRES), a Kozak sequence, a coding RNA sequence, and a second portion of the RNA element (e.g., IRES).

In some embodiments, the effector RNA sequence comprises an internal ribosomal entry site (IRES) sequence operably linked to the coding RNA sequence.

In some embodiments, the effector RNA sequence comprises an m6A modification motif sequence operably linked to the coding RNA sequence.

In some embodiments, the linear RNA further comprises a polyA or polyAC sequence disposed at the 3′ end of the coding RNA sequence and at the 5′ end of the second portion of the RNA element (e.g., IRES). The internal polyA sequence or polyAC spacer may range from 1 to 500 nucleotides in length (e.g., at least 20, 30, 40, 50, 60, 70, 80, 90, 120, 140, 160, 180, 200, 250, 300, 350, 400, 450, or 500 nucleotides). In some embodiments, the polyA sequence or polyAC sequence may range from 10-70, 20-60, or 30-60 nucleotides in length. In some embodiments, the linear RNA comprises no polyA sequence or polyAC sequence. Without being bound by any theory or hypothesis, an internal polyA sequence or a polyAC spacer added before IRES sequences in a circRNA can help to keep the functional second structure of IRES elements for efficient protein translation initiated by IRES. In some embodiments, the polyA sequence or polyAC spacer increases expression of the coding RNA.

In some embodiments, the effector RNA sequence comprises a nucleic acid sequence comprising a therapeutic RNA. In some embodiments, the therapeutic RNA is an RNA molecule selected from the group consisting of a gRNA, a dRNA, a siRNA, a miRNA, a shRNA, and a lincRNA.

### III. Circular RNAs and Methods of Use

The present application further provides circRNAs and compositions prepared using any one of the methods of preparation described herein.

In some embodiments, the circRNA comprises an effector RNA sequence. In some embodiments, the effector RNA sequence is a coding RNA. In some embodiments, the effector RNA sequence comprises a nucleic acid sequence encoding a therapeutic polypeptide. In some embodiments, the therapeutic polypeptide is selected from the group consisting of an antigenic polypeptide, a functional protein, a receptor protein, and a targeting protein.

In some embodiments, the effector RNA sequence comprises a Kozak sequence operably linked to the to the nucleic acid sequence encoding the therapeutic polypeptide.

In some embodiments, the effector RNA sequence comprises an in-frame 2A peptide coding sequence operably linked to the 3′ end of the nucleic acid sequence encoding the therapeutic polypeptide.

In some embodiments, the effector RNA sequence comprises an internal ribosomal entry site (IRES) sequence operably linked to the nucleic acid sequence encoding the therapeutic polypeptide.

In some embodiments, the effector RNA sequence comprises an m6A modification motif sequence operably linked to the nucleic acid sequence encoding the therapeutic polypeptide.

In some embodiments, the effector RNA sequence comprises a nucleic acid sequence comprising a therapeutic RNA. In some embodiments, the therapeutic RNA is an RNA molecule selected from the group consisting of a gRNA, a dRNA, a siRNA, a miRNA, a shRNA, and a lincRNA.

In some embodiments, there is provided a cocktail composition comprising a plurality of circRNAs each comprising a coding RNA sequence encoding an antigenic polypeptide, a receptor protein of an infectious agent, or a targeting protein (e.g., an antibody such as a neutralizing antibody). In some embodiments, the plurality of circRNA encode antigenic polypeptides that are different with respect to each other, such as different mutants of an antigenic polypeptide (e.g., S protein or fragment thereof). In some embodiments, the plurality of circRNA encode receptor proteins that are different with respect to each other, such as different mutants of a receptor protein (e.g., ACE2). In some embodiments, the plurality of circRNA encode targeting proteins that are different with respect to each other, such as different antibodies (e.g., neutralizing antibodies).

The circRNAs described herein may be used to treat or prevent a disease or condition in an individual, including, but not limited to genetic diseases (e.g., hereditary genetic diseases, metabolic diseases and cancer), and infections (e.g., viral infections such as coronavirus infections). In some embodiments, the circRNA is subject to rolling circle translation by a ribosome in the individual.

In some embodiments, there is provided a method of treating or preventing a disease or condition in an individual, comprising administering to the individual an effective amount of a circRNA prepared using any one of the methods described herein. In some embodiments, the circRNA comprises a coding RNA sequence encoding a functional protein. In some embodiments, the functional protein is an enzyme, a receptor, a ligand, a signaling molecule, or a transcription factor. In some embodiments, the disease or condition is a metabolic disease. In some embodiments, the disease or condition is a lysosomal storage disorder. In some embodiments, the disease or condition is a cancer.

The circRNAs described herein may be used for treating a genetic disease or condition that is associated with a mutation or deficiency in a naturally-occurring protein corresponding to the therapeutic polypeptide encoded by the circRNA. In some embodiments, the disease or condition is a disease or condition associated with insufficient levels and/or activity of a naturally-occurring protein corresponding to the therapeutic polypeptide. In some embodiments, the disease or condition is a hereditary genetic disease associated with one or more mutations in naturally-occurring protein corresponding to the therapeutic polypeptide. In some embodiments, the therapeutic polypeptide is a wildtype protein, or a functional variant thereof (e.g., a functional fragment, fusion protein, or mutant).

In some embodiments, the therapeutic polypeptide can be any polypeptide that is capable of being expressed by target cells (e.g., human or mouse cells) for the production (and in certain instances, the excretion) of a functional enzyme or protein as disclosed, for example, in International Application No. PCT/US2010/058457. In some embodiments, the therapeutic polypeptide can be engineered for secretion by operably linking a signal peptide to the amino terminus of the therapeutic polypeptide. For example, in some embodiments, upon the expression of one or more therapeutic polynucleotides by target cells, the production of a functional enzyme or protein in which a subject is deficient (e.g., a urea cycle enzyme or an enzyme associated with a lysosomal storage disorder) may be observed.

Examples of disease-associated mutations that may be treated by the methods of the present application include, but are not limited to, TP53^{W53X} (e.g., 158G>A) associated with cancer, IDUA^{W402X} (e.g., TGG>TAG mutation in exon 9) associated with Mucopolysaccharidosis type I (MPS I), COL3A1^{W1278X} (e.g., 3833G>A mutation) associated with Ehlers-Danlos syndrome, BMPR2^{W298X} (e.g., 893G>A) associated with primary pulmonary hypertension, AHI1^{W725Z} (e.g., 2174G>A) associated with Joubert syndrome, FANCC^{W506X} (e.g., 1517G>A) associated with Fanconi anemia, MYBPC3^{W1098X} (e.g., 3293G>A) associated with primary familial hypertrophic cardiomyopathy, and IL2RG^{W237X} (e.g., 710G>A) associated with X-linked severe combined immunodeficiency. In some embodiments, the disease or condition is a cancer. In some embodiments, the disease or condition is a monogenetic disease. In some embodiments, the disease or condition is a polygenetic disease.

In some embodiments, the circRNA has a functional half-life of at least or at least about 20 hours, 24 hours, 30 hours, or 36 hours. In some embodiments, the circRNA has a duration of therapeutic effect in a human cell of at least or at least about 20 hours, 24 hours, 30 hours, or 36 hours. In some embodiments, the circRNA has a duration of therapeutic effect in a human cell greater than or equal to that of an equivalent linear RNA comprising the same expression sequence. In some embodiments, the circRNA has a functional half-life in a human cell greater than or equal to that of an equivalent linear RNA comprising the same expression sequence.

In some embodiments, the present application provides circRNAs for use in treating or preventing a disease or condition in an individual.

In some embodiments, the present application provides use of a circRNA comprising a nucleic acid sequence encoding a therapeutic polypeptide for the manufacture of a medicament for treating or preventing a disease or condition in an individual.

In some embodiments, the circRNA is administered as naked circRNA, or as a pharmaceutical composition comprising a transfection agent. In non-limiting examples, the transfection agent is polyethylenimine (PEI) or a lipid nanoparticle (LNP). Other examples of lipidosomes that can be used to administer the circRNA composition for administration (e.g., circRNA vaccine or pharmaceutical composition) include protamines, cationic nanoemulsions, modified dendrimer nanoparticles, protamine liposomes, cationic polymers, cationic polymer liposomes, polysaccharide particles, cationic lipid nanoparticles, cationic lipid-cholesterol nanoparticles, cationic lipid-cholesterol PEG nanoparticle, cationic lipid transfection reagents sold under the trademark LIPOFECTAMINE, nonliposomal transfection reagents sold under the trademark FUGENE, or any combination thereof can be used as the transfection agent.

In some embodiments, the liposome formulation may be influenced by, but not limited to, the selection of the cationic lipid component, the degree of cationic lipid saturation, the nature of the PEGylation, ratio of all components and biophysical parameters such as size. In some embodiments, the liposome formulation comprises a cationic lipid, a cholesterol and a PEGylated lipid. For example, a liposome formulation may comprise a cationic lipid, dipalmitoylphosphatidylcholine, cholesterol, and PEG-c-DMA. See, for example, Semple et al. Nature Biotech. 2010 28:172-176, herein incorporated by reference in its entirety. In some embodiments, liposome formulations may comprise from about 35 to about 45% cationic lipid, from about 40% to about 50% cationic lipid, from about 50% to about 60% cationic lipid and/or from about 55% to about 65% cationic lipid. In some embodiments, the ratio of lipid to RNA in liposomes may be from about 5:1 to about 20:1, from about 10:1 to about 25:1, from about 15:1 to about 30:1 and/or at least 30:1. Suitable liposome formulations have been described, for example, in WO2020237227, the contents of which are herein incorporated by reference in their entirety.

In some embodiments, the circRNA is not formulated with a transfection reagent. In some embodiments, the circRNA is delivered as naked RNA. In some embodiments, the circRNA is delivered by gene gun or by electroporation.

The circRNA composition for administration (e.g., circRNA vaccine or pharmaceutical composition) can be administered to a subject by systemic injection into the vasculature, systemic injection into the lymph nodes, subcutaneous injection or depots, or by local injection.

In some embodiments, the circRNA may be formulated in a lipid nanoparticle such as those described in International Publication No. WO2012170930, herein incorporated by reference in its entirety.

In some embodiments, the synthetic nanocarriers may be formulated for controlled and/or sustained release of the circRNA described herein. As a non-limiting example, the synthetic nanocarriers for sustained release may be formulated by methods known in the art, described herein and/or as described in International Pub No. WO2010138192 and US Pub No. 20100303850, each of which is herein incorporated by reference in their entirety.

In some embodiments, the circRNA may be formulated for controlled and/or sustained release wherein the formulation comprises at least one polymer that is a crystalline side chain (CYSC) polymer. CYSC polymers are described in U.S. Pat. No. 8,399,007, herein incorporated by reference in its entirety.

In some embodiments, the synthetic nanocarrier may be formulated for use as a vaccine. In some embodiments, the synthetic nanocarrier may encapsulate at least one circRNA, which encode at least one antigen. As a nonlimiting example, the synthetic nanocarrier may include at least one antigen and an excipient for a vaccine dosage form (see International Pub No. WO2011150264 and US Pub No. US201 10293723, each of which is herein incorporated by reference in their entirety). As another non-limiting example, a vaccine dosage form may include at least two synthetic nanocarriers with the same or different antigens and an excipient (see International Pub No. WO201 1150249 and US Pub No. US201 10293701, each of which is herein incorporated by reference in their entirety). The vaccine dosage form may be selected by methods described herein, known in the art and/or described in International Pub No. WO2011150258 and US Pub No. US20120027806, each of which is herein incorporated by reference in their entirety).

In some embodiments, the synthetic nanocarrier may comprise at least one circRNA, which encodes at least one adjuvant. As non-limiting example, the adjuvant may comprise dimethyldioctadecylammonium-bromide, dimethyldioctadecylammoniumchloride, dimethyldioctadecylammonium-phosphate or dimethyldioctadecylammoniumacetate (DDA) and an apolar fraction or part of said apolar fraction of a total lipid extract of a mycobacterium (See e.g., U.S. Pat. No. 8,241,610; herein incorporated by reference in its entirety). In another embodiment, the synthetic nanocarrier may comprise at least one circRNA and an adjuvant. As a non-limiting example, the synthetic nanocarrier comprising and adjuvant may be formulated by the methods described in International Pub No. WO2011150240 and US Pub No. US20110293700, each of which is herein incorporated by reference in its entirety.

In some embodiments, the circRNA functions as an adjuvant. As an example, RNA-sensing in the cytoplasm can trigger innate immunity, and innate immune signaling is known to contribute to adaptive immunity by diverse routes. Thus, the circRNA encoding the antigenic polypeptide or a second circRNA (e.g., a circRNA that does not encode a polypeptide) can be used as an adjuvant for boosting the adaptive immune response to the antigenic polypeptide.

In some embodiments, the circRNA compositions of the present application may be administrated with other prophylactic or therapeutic compounds. As a non-limiting example, the prophylactic or therapeutic compound may be an adjuvant or a booster. As used herein, when referring to a prophylactic composition, such as a vaccine, the term “booster” refers to an extra administration of the prophylactic composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years.

In some embodiments, the circRNA composition for administration (e.g., circRNA vaccine or pharmaceutical composition) may be administered intranasally. For example, circRNA vaccines may be administered intranasally similar to the administration of live vaccines. In some embodiments, the circRNA may be administered intramuscularly or intradermally similarly to the administration of inactivated vaccines known in the art.

In some embodiments, the circRNA vaccine comprises an adjuvant, which may enable the vaccine to elicit a higher immune response. As a non-limiting example, the adjuvant could be a sub-micron oil-in-water emulsion, which can elicit a higher immune response in human pediatric populations (see e.g., the adjuvant-containing vaccines described in US Patent Publication No. US20120027813 and U.S. Pat. No. 8,506,966, the contents of each of which are herein incorporated by reference in its entirety).

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended embodiments.

### Example 1. In Vitro Single-Pot Preparation of circRNA Group I Ribozyme Autocatalysis

This example demonstrates in vitro single-pot preparation of a circular RNA (circRNA) by Group I ribozyme autocatalysis.

A linear RNA was designed that can be circularized to produce a circRNA comprising, from 5′ to 3′, a 5′Homology arm-3′ catalytic Group I intron fragment-3′ exon sequence recognizable by the 3′ catalytic Group I intron fragment (i.e., Exon 2)-m6A modification motif-Kozak-SP-Spike-2A peptide-5′ exon sequence recognizable by the 5′ catalytic Group I intron fragment (i.e., Exon 1)-5′ catalytic Group I intron fragment-3′Homology arm, as shown in FIG. 1. The linear RNA is designed with, from 5′ to 3′, a 5′ homology arm (SEQ ID NO: 5), a 3′ catalytic Group I intron sequence (SEQ ID NO: 1), a 3′ exon sequence recognizable by a 3′ catalytic Group I intron fragment (SEQ ID NO: 3), a m6A modification motif sequence (SEQ ID NO: 7), a Kozak sequence (SEQ ID NO: 8), a signal peptide coding sequence (SEQ ID NO: 12 or SEQ ID NO: 13), a Spike protein RBD sequence encoding the amino acid sequence shown in SEQ ID NO: 11, a stop codon, a 2A peptide coding sequence (SEQ ID NO: 9 or SEQ ID NO: 10), a 5′ exon sequence recognizable by a 5′ catalytic Group I intron fragment (SEQ ID NO: 4), a 5′ catalytic Group I intron fragment (SEQ ID NO: 2), and a 3′ homology arm (SEQ ID NO: 6).

Linear RNAs that can be circularized to produce the circular RNA (circRNAs) disclosed herein may be made using standard laboratory methods and materials. The cDNA sequence encoding the linear RNA may be synthesized by de novo DNA synthesis. The synthetic nucleic acid can be ordered from a synthetic nucleotide service such as GBLOCKS® (Integrated DNA Technologies). The nucleic acid sequence encoding the linear RNA sequence can be cloned into a plasmid vector containing a T7 promoter, the multiple cloning site flanked by restriction sites such as Xba1 restriction sites. The resulting plasmid may be transformed into chemically competent E. coli.

For the present example, NEB DH5-alpha Competent E. coli cells were used. Transformations were performed according to NEB instructions using 100 ng of plasmid. The protocol is as follows:
1. Thaw a tube of NEB 5-alpha Competent E. coli cells on ice for 10 minutes.
2. Add 1-5 μL containing 1 pg-100 ng of plasmid DNA to the cell mixture. Carefully flick the tube 4-5 times to mix cells and DNA. Do not vortex.
3. Place the mixture on ice for 30 minutes. Do not mix.
4. Heat shock at 42° C. for exactly 30 seconds. Do not mix.
5. Place on ice for 5 minutes. Do not mix.
6. Pipette 950 μL of room temperature SOC into the mixture.
7. Place at 37° C. for 60 minutes. Shake vigorously (250 rpm) or rotate.
8. Warm selection plates to 37° C.
9. Mix the cells thoroughly by flicking the tube and inverting.

Spread 50-100 μL of each dilution onto a selection plate and incubate overnight at 37° C. Alternatively, incubate at 30° C. for 24-36 hours or 25° C. for 48 hours.

A single colony was then used to inoculate 5 ml of LB growth media using the appropriate antibiotic and then allowed to grow (250 RPM, 37° C.) for 5 hours. This was then used to inoculate a 200 ml culture medium and allowed to grow overnight under the same conditions. To isolate the plasmid (up to 850 mg), a maxi prep was performed using the Invitrogen PURELINK™ HiPure Maxiprep Kit (Carlsbad, CA), following the manufacturer's instructions.

In order to generate a linearized plasmid DNA template for In Vitro Transcription (IVT), the plasmid was first linearized using a restriction enzyme, such as Xba1. A typical restriction digest with Xba1 comprises the following: Plasmid 1.0 mg 10× Buffer 1.0 mL; Xba1 1.5 mL; dH20 up to 10 mL; incubated at 37° C. for 1 hr. When performed at lab scale (<5), the reaction is cleaned up using Invitrogen's PURELINK™ PCR Micro Kit (Carlsbad, CA) per manufacturer's instructions. Larger scale purifications may need to be done with a product that has a larger load capacity such as Invitrogen's standard PURELINK™ PCR Kit (Carlsbad, CA). Following the cleanup, the linearized vector was quantified using the NanoDrop and analyzed to confirm linearization using agarose gel electrophoresis.

circRNA was then generated using a single-pot in vitro transcription (IVT) process without the need of DNase I treatment or additional GTP treatment. Unmodified linear RNA precursors were synthesized by in-vitro transcription from a linearized plasmid DNA template using a T7 High Yield RNA Synthesis Kit (New England Biolabs). During the in vitro transcription (IVT) process, the linear RNA precursor are catalyzed into circRNA products through the self-splicing of the Group I intron in the presence of GTPs, which exist in the IVT reaction system. To enrich for circRNA, 20 μg of RNA was diluted in water (86 μL final volume) and then heated at 65° C. for 3 min and cooled on ice for 3 min. 20U RNase R and 10 μL of 10×RNase R buffer (Epicenter) was added, and the reaction was incubated at 37° C. for 15 min. RNase R-digested RNA is column purified.

Previous circRNA methods have required DNase I treatment and/or additional GTP treatment. As a comparison to the single-pot method described above, circular RNA was prepared using a multi-step method according to the following steps. The method used unmodified linear mRNA or circRNA precursors synthesized by in vitro transcription from a linearized plasmid DNA template using a T7 High Yield RNA Synthesis Kit (New England Biolabs). After in vitro transcription, the reaction product was treated with DNase I (New England Biolabs) for 20 min. After DNase treatment, unmodified linear nNA was column purified using a MEGAclear Transcription Clean-up kit (Ambion). For circularization of the linear RNA precursor, additional GTP was added to a final concentration of 2 mM, and then reactions were heated at 55° C. for 15 min. RNA was then column purified. In some cases, purified RNA was re-circularized: RNA was heated to 70° C. for 5 min and then immediately placed on ice for 3 min, after which GTP was added to a final concentration of 2 mM along with a buffer including magnesium (50 mM Tris-HCl, 10 mM MgCl2, 1 mM DTT, pH 7.5; New England Biolabs). RNA was then heated to 55° C. for 8 min, and then column purified. To enrich for circRNA, 20 μg of RNA was diluted in water (86 μL final volume) and then heated at 65° C. for 3 min and cooled on ice for 3 min. 2U RNase R and 10 μL of 10× RNase R buffer (Epicenter) was added, and the reaction was incubated at 37° C. for 15 min.

For gel extractions, bands corresponding to the circRNA were excised from the gel and then extracted using a Zymoclean Gel RNA Extraction Kit (Zymogen). For high-performance liquid chromatography, 30 μg of RNA was heated at 65° C. for 3 min and then placed on ice for 3 min. RNA was run through a 4.6×300 mm size-exclusion column with particle size of 5 μm and pore size of 2000 Å (Sepax Technologies; part number: 215980P-4630) on an Agilent 1100 Series HPLC (Agilent). RNA was run in RNase-free TE ss (10 mM Iris, 1 mM EDTA, pH:6) at a flow rate of 0.3 mL/minute. RNA was detected by UV absorbance at 260 nm. Resulting RNA fractions are precipitated with 5 M ammonium acetate, resuspended in water, and then in some cases treated with RNase R as described above.

The resulting circRNA is shown in FIG. 1B.

### Example 2. In Vitro circRNA Production by Group I Ribozyme Autocatalysis

This example demonstrates circRNA can be produced in a single-pot reaction step as described herein.

First, a circRNA construct was designed comprising a nucleotide sequence encoding an RBD of a SARS-CoV-2 Spike protein, using the circRNA backbone as described in Example 1 above.

Briefly, linear RNAs were designed that can be circularized to produce a circRNA, the linear RNAs comprising, from 5′ to 3′, a 5′Homology arm-3′ catalytic Group I intron fragment-3′ exon sequence recognizable by the 3′ catalytic Group I intron fragment (i.e., Exon 2)-IRES-Kozak-SP-RBD-TAA stop codon-5′ exon sequence recognizable by the 5′ catalytic Group I intron fragment (i.e., Exon 1)-5′ catalytic Group I intron fragment-3′Homology arm. The linear RNA is designed with, from 5′ to 3′, a 5′ homology arm (SEQ ID NO: 5), a 3′ catalytic Group I intron sequence (SEQ ID NO: 1), a 3′ exon sequence recognizable by a 3′ catalytic Group I intron fragment (SEQ ID NO: 3), a m6A modification motif sequence (SEQ ID NO: 7), a Kozak sequence (SEQ ID NO: 8), a signal peptide coding sequence (SEQ ID NO: 12 or SEQ ID NO: 13), a Spike protein RBD sequence encoding the amino acid sequence shown in SEQ ID NO: 11, a stop codon, a 2A peptide coding sequence (SEQ ID NO: 9 or SEQ ID NO: 10), a 5′ exon sequence recognizable by a 5′ catalytic Group I intron fragment (SEQ ID NO: 4), a 5′ catalytic Group I intron fragment (SEQ ID NO: 2), and a 3′ homology arm (SEQ ID NO: 3). The circularized RNAs produced from this linear RNA were termed circRNA^{RBD}

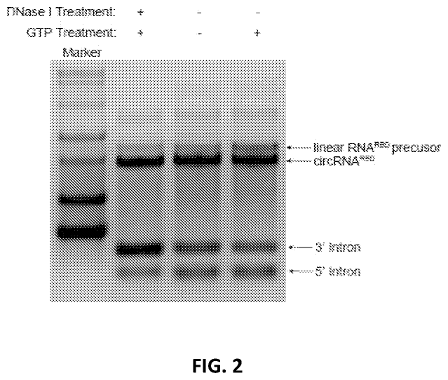

A circRNA was generated and purified as described in Example 1. As controls, circRNA was generated and purified with DNase I treatment and additional GTP treatment, or GTP treatment alone with no DNase I treatment. The purified circRNA^{RBD} were resolved in agarose gel electrophoresis. The gel electrophoresis results show that circRNA^{RBD} could be produced without requiring DNase I treatment or additional GTP treatment (FIG. 2).

EXEMPLARY SEQUENCES
3′ catalytic Group I intron fragment
SEQ ID NO: 1
AACAAUAGAUGACUUACAACUAAUCGGAAGGUGCAGAGACUCGACGGGA
GCUACCCUAACGUCAAGACGAGGGUAAAGAGAGAGUCCAAUUCUCAAAG
CCAAUAGGCAGUAGCGAAAGCUGCAAGAGAAUG
5′ catalytic Group I intron fragment
SEQ ID NO: 2
AAAUAAUUGAGCCUUAAAGAAGAAAUUCUUUAAGUGGAUGCUCUCAAAC
UCAGGGAAACCUAAAUCUAGUUAUAGACAAGGCAAUCCUGAGCCAAGCC
GAAGUAGUAAUUAGUAAG
3′ exon sequence recognizable by a 3′ catalytic
Group I intron fragment
SEQ ID NO: 3
AAAAUCCGUUGACCUUAAACGGUCGUGUGGGUUCAAGUCCCUCCACCCC
CAC
5′ exon sequence recognizable by a 5′ catalytic
Group I intron fragment
SEQ ID NO: 4
GAGACGCUACGGACUU
Exemplary 5′ homology sequence
SEQ ID NO: 5
GGGAGACCCUCGACCGUCGAUUGUCCACUGGUC
Exemplary 3′ homology sequence
SEQ ID NO: 6
ACCAGUGGACAAUCGACGGAUAACAGCAUAUCUAG
m6A modification sequence
SEQ ID NO: 7
ACGAGTCCTGGACTGAAACGGACTTGT
Kozak nucleic acid sequence
SEQ ID NO: 8
GCCACCAUG
T2A peptide coding sequence
SEQ ID NO: 9
GAGGGCAGAGGAAGUCUUCUAACAUGCGGUGACGUGGAGGAGAAUCCCG
GCCCU
P2A peptide coding sequence
SEQ ID NO: 10
GCUACUAACUUCAGCCUGCUGAAGCAGGCUGGAGACGUGGAGGAGAACC
CUGGACCU
RBD amino acid residues 319-542 of S protein
SEQ ID NO: 11
RVQPTESIVRFPNITNLCPFGEVENATRFASVYAWNRKRISNCVADYSV
LYNSASFSTFKCYGVSPTKLNDLCFTNVYADSFVIRGDEVRQIAPGQTG
KIADYNYKLPDDFTGCVIAWNSNNLDSKVGGNYNYLYRLFRKSNLKPFE
RDISTEIYQAGSTPCNGVEGENCYFPLQSYGFQPTNGVGYQPYRVVVLS
FELLHAPATVCGPKKSTNLVKNKCVNF
tissue plasminogen activator SP
SEQ ID NO: 12
DAMKRGLCCVLLLCGAVFVSPSQEIHARFRR
human IgE immunoglobulin SP
SEQ ID NO: 13
DWTWILFLVAAATRVHS

1. A method of producing a circular RNA from a DNA construct encoding a linear RNA precursor, wherein the linear RNA precursor comprises from the 5′-end to the 3′ end: a 3′ catalytic Group I intron fragment, a 3′ exon sequence, an effector RNA sequence, a 5′ exon sequence, and a 5′ catalytic Group I intron fragment, wherein the method comprises an in vitro single-pot reaction step comprising contacting the DNA construct with a reagent composition comprising an RNA polymerase, adenosine 5′-triphosphate (ATP), uridine 5′-triphosphate (UTP), guanosine 5′-triphosphate (GTP) and cytosine 5′-triphosphate (CTP) under conditions that allow transcription of the DNA construct into the linear RNA precursor and circularization of the linear RNA precursor, wherein the circularization comprises activation of the 3′ catalytic Group I intron fragment and the 5′ catalytic Group I intron fragment to splice the 3′ exon sequence and the 5′ exon sequence from the linear RNA precursor, thereby forming the circular RNA comprising the effector RNA.

2. The method of claim 1, wherein the single-pot reaction step does not comprise supplementing the reagent composition with guanosine 5′-triphosphate (GTP) prior to the circularization of the linear RNA precursor.

3. The method of claim 1, wherein the single-pot reaction step does not comprise supplementing the reagent composition with a divalent metal ion prior to the circularization of the linear RNA precursor.

4-5. (canceled)

6. The method of claim 1, wherein the single-pot reaction step does not comprise DNAse I treatment prior to the circularization of the linear RNA precursor.

7. The method of claim 1, wherein the RNA polymerase is T7 RNA polymerase.

8. The method of claim 1, wherein the reagent composition comprises about 0.01 mM to about 50 mM of each of ATP, UTP, GTP and CTP.

9. (canceled)

10. The method of claim 1, wherein the DNA construct is contacted with the reagent composition for at least about 20 minutes.

11. The method of claim 1, wherein the DNA construct is a plasmid.

12-15. (canceled)

16. The method of claim 1, wherein the 3′ catalytic Group I intron fragment and the 5′ catalytic Group I intron fragment are derived from Anabaena Group I intron.

17. The method of claim 16, wherein the 3′ catalytic Group I intron fragment comprises the nucleic acid sequence of SEQ ID NO: 1, and the 5′ catalytic Group I intron fragment comprises the nucleic acid sequence of SEQ ID NO: 2.

18. The method of claim 1, wherein the 3′ exon sequence comprises the nucleic acid sequence of SEQ ID NO: 3, and the 5′ exon sequence comprises the nucleic acid sequence of SEQ ID NO: 4.

19. The method of claim 1, wherein the linear RNA precursor further comprises a 5′ homology arm sequence flanking the 5′ of the 3′ catalytic Group I intron fragment, and a 3′ homology arm sequence flanking the 3′ of the 5′ catalytic Group I intron fragment, wherein the 5′ homology arm sequence and the 3′ homology arm sequence hybridize with each other.

20. The method of claim 19, wherein the 5′ homology arm sequence and the 3′ homology arm sequence are each about 5 to 100 nucleotides in length.

21. (canceled)

22. The method of claim 1, wherein the effector RNA sequence comprises a nucleic acid sequence encoding a therapeutic polypeptide.

23. (canceled)

24. The method of claim 22, wherein the effector RNA sequence comprises a Kozak sequence operably linked to the to the nucleic acid sequence encoding the therapeutic polypeptide.

25. The method of claim 22, wherein the effector RNA sequence comprises an in-frame 2A peptide coding sequence operably linked to the 3′ end of the nucleic acid sequence encoding the therapeutic polypeptide.

26. The method of claim 22, wherein the effector RNA sequence comprises an internal ribosomal entry site (IRES) sequence operably linked to the nucleic acid sequence encoding the therapeutic polypeptide.

27. The method of claim 22, wherein the effector RNA sequence comprises an m6A modification motif sequence operably linked to the nucleic acid sequence encoding the therapeutic polypeptide.

28. The method of claim 1, wherein the effector RNA sequence comprises a nucleic acid sequence comprising a therapeutic RNA.

29. A circular RNA prepared using the method of claim 1.
